(19) United States
(12) Patent Application Publication
(10) Pub. No.: US 2023/0134410 A1
(43) Pub. Date: May 4, 2023

(54) Isolator

(71) Applicant: Howorth Air Technology Limited, Bolton (GB)

(72) Inventors: Michael MELLOR, Greater Manchester (GB); Graham HIND, Greater Manchester (GB)

(21) Appl. No.: 17/794,122

(22) PCT Filed: Jan. 22, 2021
(86) PCT No.: PCT/GB2021/050142

§ 371 Date: Jul. 20, 2022

(30) Foreign Application Priority Data
Jan. 24, 2020 (GB) 2001007.0

(51) Int. Cl. B01L 1/04; B25J 21/02

(52) U.S. Cl. CPC B01L 1/04; B25J 21/02; B01L 2200/028; B01L 2200/082; B01L 2200/085

(57) ABSTRACT

The disclosure provides a modular isolator apparatus. The apparatus comprises an isolator comprising a housing, having an opening therein, and an isolator coupling member disposed around the opening. The apparatus further comprises at least one functional unit comprising a functional unit coupling member configured to reversibly couple with the isolator coupling member, thereby reversibly coupling the functional unit and the isolator. The apparatus further comprises at least one sealing member, configured to create an airtight seal between the isolator and functional unit coupling members when they are coupled; and at least one locking member, configured to secure the functional unit to the isolator when the isolator and functional unit are coupled. The apparatus is characterised in that when the functional unit is not secured to the isolator it is removable therefrom.

The present invention relates to isolators, and in particular to a modular isolator apparatus.

An isolator or a glovebox (glove box) is a sealed chamber that allows for the handling of objects where an isolated environment is required. Gloves are built into the isolator/glovebox and placed so that that the user can insert their hands into the gloves and perform tasks inside the chamber without breaking containment. Isolators are sealed structures with filtration systems allowing the handling of hazardous and toxic substances. All exhaust air leaving the isolator is filtered with a high-efficiency particulate air filter or HEPA, thereby removing harmful toxic powders bacteria and viruses.

Alternatively, or additionally, isolators can provide an aseptic environment to protect a product and/or materials from the external environments.

Special consideration is needed to allow objects and materials to be passed into and out of the sealed isolator/glovebox without breaking the containment and/or the aseptic environment. An isolator/glovebox may have either positive, neutral or negative internal pressures to assist with the required protection to either the operators and/or the products.

An isolator may comprise additional features that provide added functionality. For instance, some isolators comprise a rapid transfer port (RTP), allowing hazardous material to be rapidly transferred in and out of the isolator. Some isolators comprise a pass-through hatch end airlock, which also allows material to be transferring in and out of the isolator. Alternatively, or additionally, multiple isolators may be interconnected.

However, retrofitting these features to an existing isolator may be difficult and can prove costly. Furthermore, a user may require multiple different isolators, each with different functionality, for different applications.

The present invention arose from the inventors’ work in attempting to address the problems associated with the prior art.

In accordance with a first aspect of the invention, there is provided a modular isolator apparatus comprising:
an isolator comprising a housing, having an opening therein, and an isolator coupling member disposed around the opening;
at least one functional unit comprising a functional unit coupling member configured to reversibly couple with the isolator coupling member, thereby reversibly coupling the functional unit and the isolator;
at least one sealing member, configured to create an airtight seal between the isolator and functional unit coupling members when they are coupled; and
at least one locking member, configured to secure the functional unit to the isolator when the isolator and functional unit are coupled;
characterised in that when the functional unit is not secured to the isolator it is removable therefrom.

Advantageously, different functional units with different functionality may be provided. Accordingly, the isolator may be adapted for a specific use by locating a desired functional unit thereon.

The housing may define a workspace, or chamber, therein. The housing may define a front, configured to allow a user access to the workspace during use of the isolator. The front may comprise at least one set of gloves or a glove port, configured to allow a user access to the workspace during use of the isolator.

The housing may define a first side and a second side, with the front extending therebetween. The housing may define an opening in the first side. Alternatively, the housing may define an opening in each side.

Accordingly, in some embodiments, the apparatus comprises:
an isolator comprising a housing, having two openings therein, and two isolator coupling members, each isolator coupling member being disposed around a corresponding opening;
at least two functional units, each unit comprising a functional unit coupling member configured to reversibly couple with an isolator coupling member, and thereby reversibly coupling the functional units and the isolator;
at least two sealing members, configured to create airtight seals between the isolator coupling members and functional unit coupling members when they are coupled; and
at least two locking members, configured to secure the functional units to isolator when the isolator and functional units are coupled;
characterised in that when the functional units are not secured to the isolator they are removable therefrom.

Preferably, in embodiments where the apparatus comprises more than one coupling member, each functional unit coupling member may couple with any of the isolator coupling members.

The or each locking member may independently be disposed on either the functional unit or on the isolator. In some embodiments, the or each locking member is disposed on the isolator.

The apparatus may comprise an isolator controller. The isolator controller is preferably an electronic controller and more preferably is a programmable logic controller. Preferably, the isolator comprises the isolator controller.

In some embodiments, the at least one locking member comprises at least one first lock. More preferably, the at least one locking member comprises a plurality of spaced apart first locks. The or each first lock may be configured to be controlled by a user. The or each first lock may be a manual lock. Alternatively, the or each first lock may be an automatic lock configured to lock when the user inputs an instruction to the isolator controller. The or each automatic lock may be an electronic or pneumatic lock. The plurality of spaced apart first locks may consist of between 2 and 10 first locks, more preferably between 3 and 5 first locks and most preferably 4 spaced apart first locks. The or each first lock may be a cam lock. The or each first lock may be disposed on the isolator, and most preferably on the isolator coupling member.

In some embodiments, the at least one locking member comprises at least one second lock. The or each second lock may be an automatic lock. The or each automatic lock may be an electronic or pneumatic lock. The or each second lock may be a pneumatic cylinder comprising a bolt. The or each second lock may controlled by the isolator controller. More preferably, the at least one locking member comprises a plurality of spaced apart second locks. The plurality of spaced apart second locks may consist of between 2 and 10 second locks, more preferably between 2 and 5 second locks and most preferably 2 spaced apart second locks. The or each second lock may be disposed on the isolator. Preferably, the or each second lock is disposed adjacent to the isolator coupling member.

In some embodiments, the apparatus comprises a plurality of locking members. In a preferred embodiment, the plurality of locking members comprises at least one first lock and at least one second lock. More preferably, the plurality of locking members comprises a plurality of first locks and a plurality of second locks.

The apparatus may comprise at least one sensing units connected to the isolator controller. The at least one sensing units may be configured to sense when the functional unit is coupled to the isolator. The at least one sensing units may be configured to sense when the or each first lock is locked. The at least one sensing unit may comprise a magnetically coded sensor and/or a proximity sensor. The sensing unit may be configured to detect the type of functional unit coupled to the isolator.

The isolator controller may be configured to engage the at least one second lock when the at least one sensing units sense that the functional unit is coupled to the isolator and/or the or each first lock is locked. The isolator controller may be configured to engage the at least one second lock when the at least one sensing units sense that the functional unit is coupled to and the isolator and the or each first lock is locked. The isolator controller may be configured to not disengage the at least one second lock until a user input confirms that the isolator has been fully cleaned. Advantageously, this prevents the functional unit from being removed from the isolator prior to the isolator having been cleaned. This guards against the accidental release of a contaminant from the isolator or the breaking of an aseptic environment within the isolator.

It may be appreciated that the functional unit may be configured such that, when it is coupled to the isolator, the functional unit coupling member contacts the isolator coupling member continuously around the opening.

The functional unit coupling member may comprise a first side, configured to contact a first side of the isolator coupling member when it is disposed thereon. The functional unit coupling member may comprise a flange which defines the first side of the functional unit coupling member. The first side of the isolator coupling member and the first side of the functional unit coupling member may both be substantially flat.

The functional unit coupling member may be configured to receive a portion of the or each first lock. Preferably, the flange is configured to receive a portion of the or each first lock. The flange may comprise a second side, substantially opposite the first side, wherein the second side of the flange may comprise a depression configured to receive a portion of the first lock. Preferably, the flange comprises a plurality of spaced apart depressions, wherein each depression is configured to receive a portion of a corresponding first lock. Accordingly, in embodiments where the first lock is a cam lock, the depression may be configured to receive a portion of a cam.

The functional unit coupling member may be configured to receive a portion of the or each second lock. Preferably, the flange is configured to receive a portion of the or each second lock. The function unit coupling member may define a recess in the functional unit configured to a portion of the or each second lock. Preferably, the functional unit coupling member comprises a plurality of recesses in the functional unit, wherein each recess is configured to receive a portion of a corresponding second lock. Preferably, the or each recess is provided in the flange. The or each recess may be provided in a third side of the flange, wherein the third side of the flange extends between the first and second sides. In embodiments where the second lock is a pneumatic cylinder the portion of the or each second lock may comprise the bolt.

The isolator coupling member may comprise an isolator collar. The isolator collar may define a boundary around the opening.

The isolator coupling member may comprise a second side. The second side may be substantially perpendicular to the first side. In embodiments where the isolator coupling member comprises an isolator collar, the second side may be an internal side of the isolator collar. The second side of the isolator, or internal side of the isolator collar, may be disposed substantially adjacent to the opening.

The isolator coupling member may comprise a third side. The third side may be substantially parallel to the second side. In embodiments where the isolator coupling member comprises an isolator collar, the third side may be an external side of the isolator collar. The second side of the isolator, or the internal side of isolator collar, may be disposed between the opening and the third side of the isolator, or the external side of isolator collar.

The first side of the isolator coupling member may extend between the second and third sides thereof. The first side of the isolator coupling member may be substantially parallel to the portion of the housing which defines the opening.

The functional unit coupling member may comprise a functional unit collar. The functional unit collar may be disposed on the first side of the functional unit coupling member. The functional unit collar may be configured to fit over the isolator collar. Alternatively, the functional unit collar may be configured to sit within the isolator collar. Preferably, the functional unit collar is configured to sit within the isolator collar. The functional unit collar may comprise an internal side and an external side.

It may be appreciated that when the functional unit is coupled to the isolator, the at least one sealing member provides an airtight seal around the opening.

Preferably, the or each sealing member is independently disposed on the isolator coupling member or on the functional unit coupling member. In embodiments where a sealing member is disposed on the isolator coupling member, the sealing member may be configured to contact the functional unit coupling member when the functional unit is coupled to the isolator. In embodiments where a sealing member is disposed on the functional unit coupling member, the sealing member may be configured to contact the isolator coupling member when the functional unit is coupled to the isolator.

The or each sealing member may be disposed so as to be positioned between the isolator coupling member and the functional unit coupling member when the functional unit is coupled to the isolator. Accordingly, a sealing member may be disposed on the first side of the isolator coupling member or the first side of the flange. In embodiments where the functional unit collar is configured to fit over the isolator collar, a sealing member may be disposed on an external side of the isolator collar or an internal side of the functional unit collar. In embodiments where the collar of the functional unit is configured to sit within the collar of the coupling member, a sealing member may be disposed on an external side of the functional unit collar or an internal side of the isolator collar.

In some embodiments, the apparatus comprises a first sealing member and a second sealing member. Advantageously, the second sealing member provides a fall-back should the first sealing member ever be compromised. The first and second sealing members may each be disposed as defined above.

Preferably, the or each sealing member is disposed on the isolator coupling member. In a preferred embodiment, the first sealing member is disposed on an internal side of the isolator collar. In a preferred embodiment, the second sealing member is disposed on the first side of the isolator coupling member.

The or each sealing member may be an inflatable seal or an O-ring seal. The inflatable seal may be a vacuum inflatable seal or a compressed air inflatable seal. It may be appreciated that a vacuum inflatable seal is configured to maintain an inflated configuration under atmospheric pressure, and collapses when a vacuum is applied. Conversely, a compressed air inflatable seal is configured to maintain a deflated configuration under atmospheric pressure, and inflates when compressed air is applied. In a preferred embodiment, the first sealing member is an inflatable seal. In a preferred embodiment, the second sealing member is an O-ring.

In embodiments where the at least one sealing member comprises an inflatable seal, the isolator controller may be configured to inflate and/or deflate the inflatable seal. The isolator controller may be configured to inflate or deform the inflatable seal when the at least one sensing units sense that the functional unit is coupled to the isolator and the or each first lock is locked. Alternatively, the isolator controller may be configured to inflate the inflatable seal once the at least one second lock is engaged.

The apparatus may comprise a pump and/or a fan configured to create a negative or a positive pressure in the isolator. The pump may be a vacuum pump. The isolator controller may be configured to activate the pump and/or the fan once the at least one second lock is engaged. Preferably, the isolator controller is configured to activate the pump and/or the fan once the at least one second lock is engaged and the inflatable seal has been inflated. Alternatively, the apparatus may be configured to allow a user to manually activate and deactivate the pump and/or the fan.

The isolator controller may be configured to power down the apparatus. The isolator controller may be configured to power down the apparatus when it receives an input from a user. The isolator controller may be configured to not initiate powering down of the apparatus until a user has confirmed that the isolator has been cleaned. When the isolator controller powers down the apparatus it may be configured to deactivate the pump and/or fan. When the isolator controller powers down the apparatus it may be configured to deflate the inflatable seal. Preferably, the isolator controller is configured to deflate the inflatable seal after the pump and/or fan has been deactivated. When the isolator controller powers down the apparatus it may be configured to disengage the at least one automatic locks. Preferably, the isolator controller is configured to disengage the at least one automatic locks after deactivating the pump and/or fan. Preferably, the isolator controller is configured to disengage the at least one automatic locks after deflating the inflatable seal.

The apparatus may comprise at least one guide member configured to guide the functional unit into a desired position on the coupling member. The or each guide member may comprise a projection disposed on the functional unit and a corresponding recess or aperture provided in the isolator, wherein the recess is configured to receive the projection therein. Alternatively, or additionally, the or each guide member may comprise a projection disposed on the isolator and a corresponding recess or aperture provided in the functional unit, wherein the recess or aperture is configured to receive the projection therein. In a preferred embodiment, the or each guide member comprises a projection disposed on the isolator and a corresponding aperture provided in the function unit, wherein the aperture is configured to receive the projection therein. Preferably, the apertures are provided in the flange of the functional unit.

Preferably, the apparatus comprises a plurality of spaced apart guide members. Preferably, the plurality of spaced apart guide members consist of between 2 and 10 spaced apart spaced apart guide members, more preferably between 3 and 5 spaced a part guide members and most preferably 4 spaced apart guide members.

Preferably, the apparatus is configured such that when the functional unit is coupled to the isolator, the or each sealing member is disposed between the or each guide member and the opening. Advantageously, the projections, recesses and apertures will not affect the seal.

The at least one functional unit may comprise a plurality of functional units. The plurality of functional units may comprise different functional units configured to provide different functionality.

The plurality of functional units may comprise a functional portion. The functional unit coupling member may be disposed around an edge of the functional portion. The functional portion may vary between the plurality of functional units. The functional portion may be configured to cap the isolator, allow an object to be transferred into or out of the isolator and/or may be configured to reversibly connect the isolator to a second isolator. Examples of various functional units are described in more detail below.

The functional unit, or at least one of the plurality of functional units, may comprise a panel and the functional unit coupling member may be disposed around the edge of the panel. Accordingly, in some embodiments, the functional unit coupling member comprises a flange disposed around the edge of the panel. When the functional unit is coupled to the isolator, the panel may extend across the opening. The panel may not comprise any openings or access points. This functional unit may be known as a blank end cap functional unit. Conversely, the functional unit may comprise one or more of a rapid transfer port (RTP), a bag-out port and a flexible isolator port disposed in the panel. It may be appreciated that RTPs, bag-out ports and flexible isolator ports are known in the art. In embodiments where the functional unit comprises a rapid transfer port disposed in the panel, it may be known as a rapid transfer port (RTP) functional unit. In embodiments where the functional unit comprises a bag-out port, the functional unit may be known as a bag-out port end cap. In embodiments where the functional unit comprises a flexible isolator connector, the functional unit may be known as a flexible isolator connection.

The at least one functional unit may comprise at least one functional unit configured to reversibly connect the isolator to a second isolator. Accordingly, the apparatus may comprise a second isolator. The second isolators may be a flexible isolator. Alternatively, the second isolator may be as defined above.

The functional unit, or at least one of the plurality of functional units, may comprise a first functional unit coupling member configured to reversibly couple with a first isolator coupling member on a first isolator, and a second functional unit coupling member configured to reversibly couple with a second isolator coupling member on a second isolator. The first and second isolators may be as defined above. The first and second isolator coupling members may be as defined above. The first functional unit coupling member may be configured to reversibly couple with the first isolator coupling member at the same time that the second functional unit coupling member is configured to reversibly couple with the second isolator coupling member.

In some embodiments, the functional unit comprises a conduit extending between the first and second isolator coupling members.

In some embodiments, the conduit is open. Accordingly, the functional unit may be configured to allow a gas to pass freely between the first and second isolators. In this embodiment, the functional unit may be known as an isolator to isolator sleeve functional unit.

Alternatively, a panel may extend across the conduit. The panel may comprise one or more of a rapid transfer port, a bag-out port and a HEPA filter. The HEPA filter may be a cartridge HEPA filter. In embodiments where the panel comprises a bag-out port, and optionally one or more cartridge HEPA filters, the functional unit may be known as an isolator to isolator sleeve with bag-out port.

In some embodiments, the at least one functional unit configured to reversibly connect the isolator to a second isolator may comprise a centre isolator to isolator pass hatch airlock functional unit. The centre isolator to isolator pass hatch airlock functional unit may comprise a housing defining a chamber therein. The chamber may be configured to reversibly connect the first and second isolators and to allow a user access to the chamber from outside of the isolators. The housing may define a first side with a first opening therein, and a second side with a second opening therein. The first and second isolator coupling members may be disposed around the corresponding first and second openings in the housing. Preferably, the first and second sides are disposed on opposing sides of the chamber. Preferably, the first and second sides are substantially parallel. In some embodiments, the first functional unit coupling member comprises a flange disposed around the first functional unit opening and the second functional unit coupling member comprises a flange disposed around the second functional unit opening.

The centre isolator to isolator pass hatch airlock functional unit may comprise a first door, configured to reversibly cover the first functional unit opening and at least one functional unit first sealing means, configured to create an airtight seal between the first door and the functional unit housing. The centre isolator to isolator pass hatch airlock functional unit may comprise a second door, configured to reversibly cover the second functional unit opening and at least one functional unit second sealing means, configured to create an airtight seal between the second door and the functional unit housing.

The housing of the centre isolator to isolator pass hatch airlock functional unit may define an access opening. The access opening may be defined in a third side of the housing. The third side may extend between the first and second sides. The access opening may be configured to allow a user to access the chamber from outside the isolator. The centre isolator to isolator pass hatch airlock functional unit may comprise an access door, configured to reversibly cover the functional unit access opening and at least one functional unit access sealing means, configured to create an airtight seal between the access door and the functional unit housing.

In some embodiments, the at least one functional unit may comprise a pass-through hatch end airlock functional unit. The pass-through hatch end airlock functional unit may comprise a housing defining a chamber therein. The chamber may be configured to allow a user access to the chamber from outside of the isolator. The housing may define a first opening and an access opening. The first opening may be in a first side of the housing and the access opening may be in a second side of the housing. The second side may be adjacent to the first side. The second side may be substantially perpendicular to the first side. An isolator coupling members may be disposed around the first opening in the housing. In some embodiments, the functional unit coupling member comprises a flange disposed around the functional unit opening.

The centre isolator to isolator pass-through hatch end airlock functional unit may comprise a first door, configured to reversibly cover the first functional unit opening and at least one functional unit first sealing means, configured to create an airtight seal between the first door and the functional unit housing. The centre isolator to isolator pass hatch airlock functional unit may comprise an access door, configured to reversibly cover the functional unit access opening and at least one functional unit access sealing means, configured to create an airtight seal between the access door and the functional unit housing.

The pass-through hatch end airlock functional unit and/or centre isolator to isolator functional unit may comprise a filter, preferably a HEPA filter.

The pass-through hatch end airlock functional unit and/or centre isolator to isolator functional unit may comprise a functional unit controller. Alternatively, the pass-through hatch end airlock functional unit and/or centre isolator to isolator functional unit may be connectable to the isolator controller.

The at least one first functional unit sealing means, the at least one first functional unit sealing means and/or the at least one functional unit access sealing means may comprise an O-ring seal or an inflatable seal. Preferably, the at least one first functional unit sealing means, the at least one first functional unit sealing means and/or the at least one functional unit access sealing means comprises an inflatable seal. The isolator controller or the functional unit controller may be configured to inflate the or each inflatable seal. Preferably, the isolator controller or the functional unit controller is configured to inflate the or each inflatable seal when the at least one sensing unit senses that the pass-through hatch end airlock functional unit or centre isolator to isolator functional unit is coupled to the or each isolator and the or each first lock is locked. Alternatively, the isolator controller or the functional unit controller may be configured to inflate the or each inflatable seal once the at least one second lock is engaged.

Preferably, the pass-through hatch end airlock functional unit and/or centre isolator to isolator functional unit comprises at least one first functional unit locking member configured to secure the first door in a closed configuration and at least one access functional unit locking member configured to secure the access door in a closed configuration. Preferably, the centre isolator to isolator functional unit comprises at least one second functional unit locking member configured to secure the second door in a closed configuration. The at least one first functional unit locking member, at least one second functional unit locking member and/or at least one access functional unit locking member may comprise a second lock. The or each second lock may be a pneumatic cylinder.

Preferably, the isolator controller or the functional unit controller is configured to engage the at least one first functional unit locking member, at least one second functional unit locking member and/or at least one access functional unit locking member when the at least one sensing units sense that the functional unit is coupled to the or each isolator and the or each first lock is locked. Preferably, while the isolator is in use, the isolator controller or the functional unit controller is configured to only ever disengage one of the at least one first locking member and the at least one access locking member at any given time. Preferably, while the isolator is in use, the isolator controller or the functional unit controller is configured to only ever disengage one of the at least one second locking member and the at least one access locking member at any given time.

In accordance with a second aspect, there is provided an isolator comprising:
a housing, having an opening therein, and an isolator coupling member disposed around the opening, the isolator coupling member being configured to couple with a functional unit coupling area, and thereby couple the isolator to a functional unit;
at least one sealing member, disposed on the isolator coupling member and being configured to create an airtight seal between the isolator and functional unit coupling members when they are coupled; and
at least one locking member, configured to secure the functional unit to the isolator when the isolator and functional unit are coupled;
characterised in that the isolator may be disconnected from the functional unit.

The functional unit may be understood to be disconnected from the isolator when it does not contact the isolator and is not connected with it.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:-

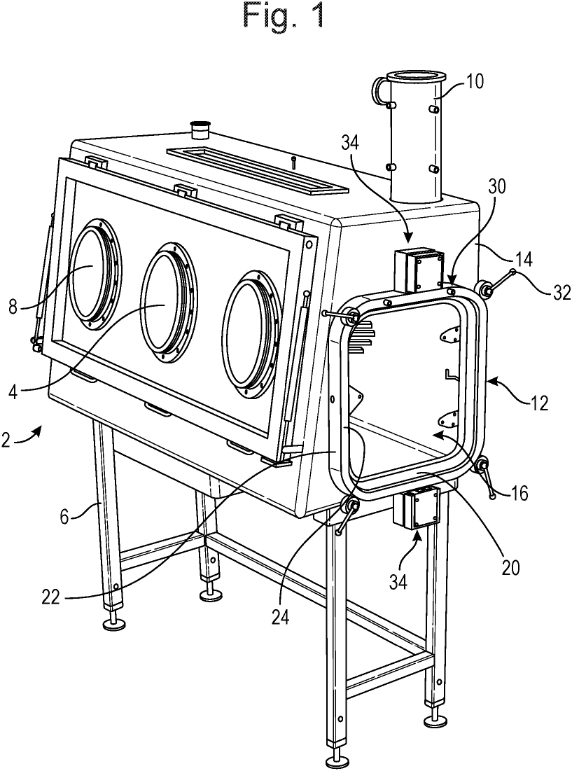

FIG. 1 is a perspective view of an isolator with a coupling member provided in a side thereof;

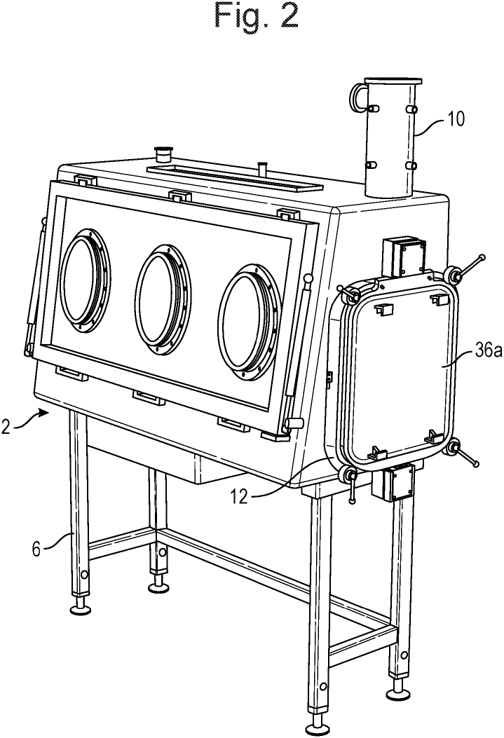

FIG. 2 is a perspective view of the isolator, wherein a blank end cap has been located over the coupling member;

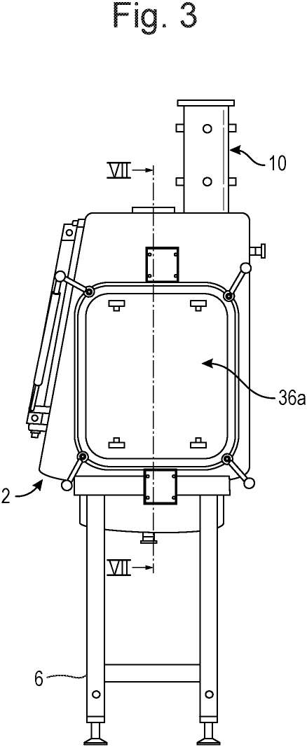

FIG. 3 is a side view of the isolator and coupling member;

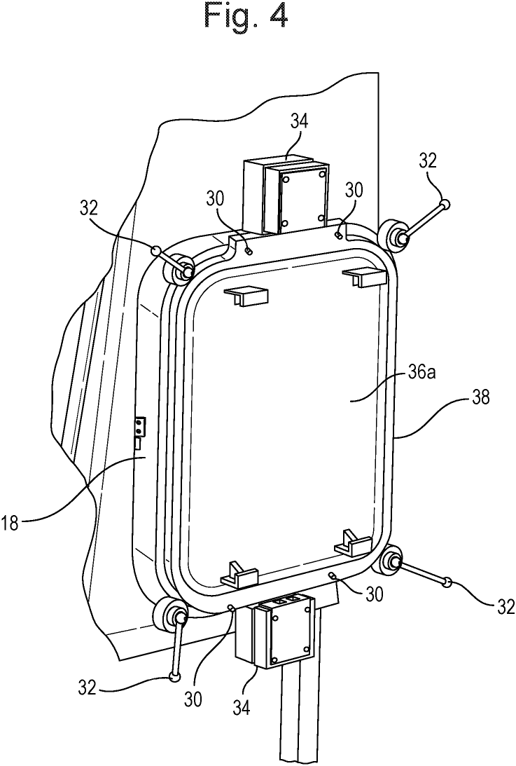

FIG. 4 is a close up of the blank end cap positioned on the isolator;

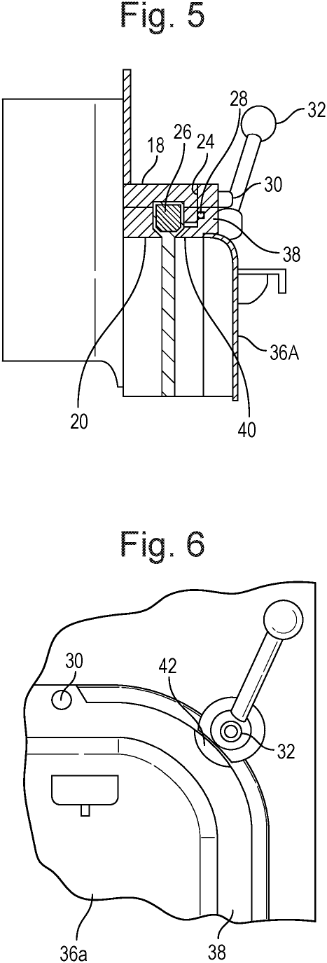

FIG. 5 is a sectional view of the isolator and blank end cap;

FIG. 6 is a close up of a portion of the blank end cap positioned on the isolator;

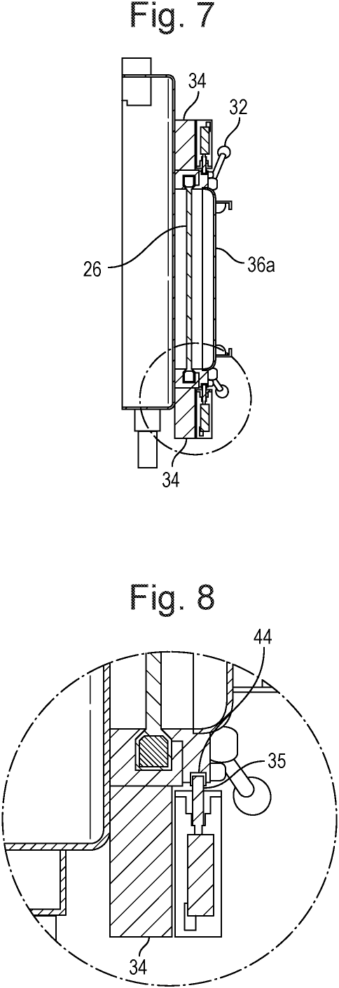

FIG. 7 is a sectional view of the isolator and blank end cap;

FIG. 8 is a close up of a portion of the sectional view of FIG. 7;

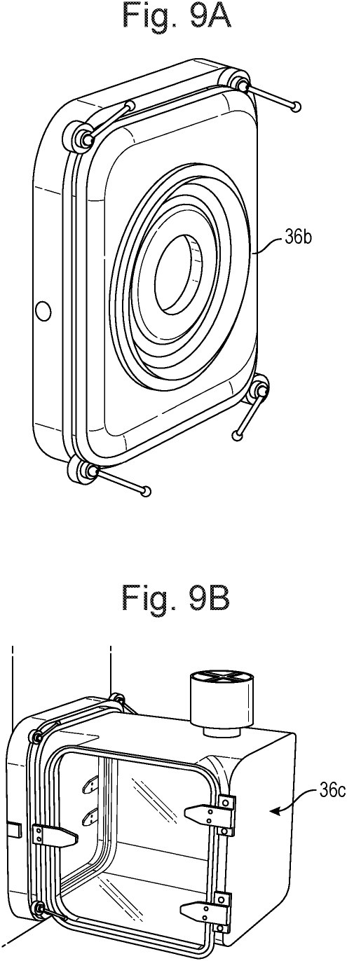

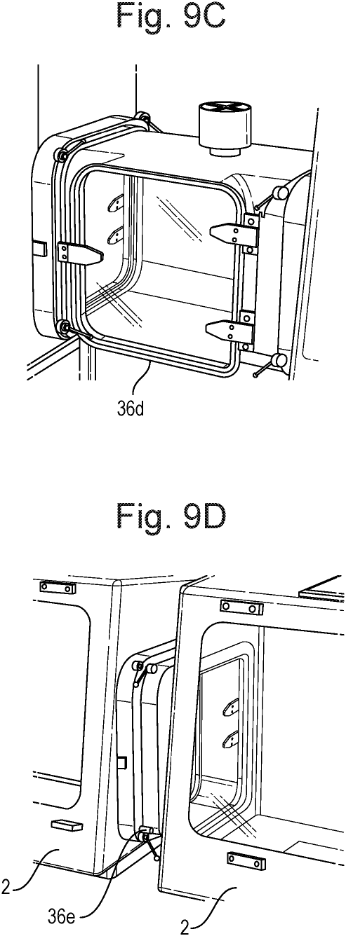

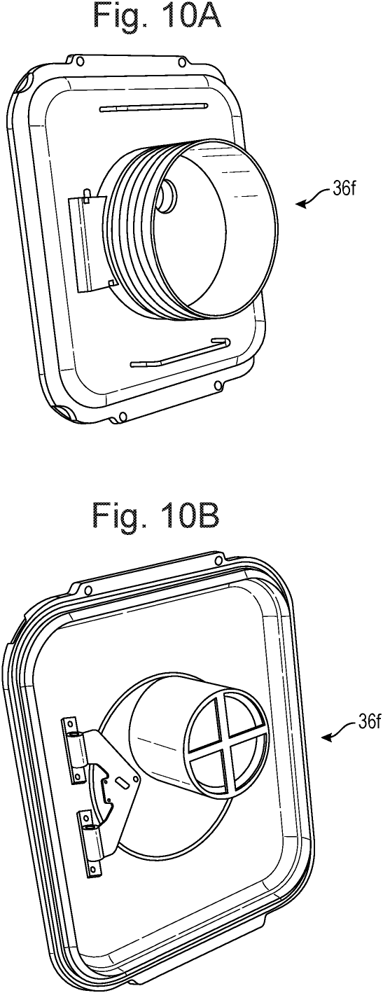

FIG. 9 shows alternative end caps which can be used with the isolator;

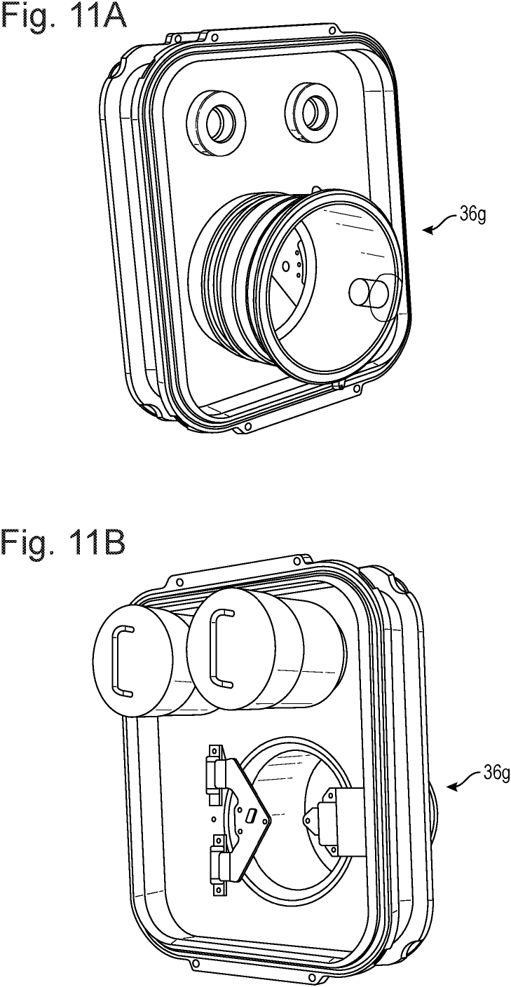

FIG. 10 shows a further alternative end cap which can be used with the isolator; and

FIG. 11 shows a still further alternative end cap which can be used with the isolator.

### EXAMPLES

Referring to FIG. 1, there is shown an isolator 2, or glovebox, configured to allow the handling of hazardous and/or toxic substances. Alternatively, or additionally, the isolator 2 can provide an aseptic environment to protect a product and/or materials therein from the external environment.

The isolator 2 includes a work area 4 provided on a support stand 6. When the isolator is in use, the work area 4, or chamber, will be fully enclosed and sealed. Three ports 8 are configured to allow gloves to be attached to the front of the isolator 2. This allows a user to access the work area 4, while preventing direct contact with any hazardous or sterile materials therein. All exhaust air leaving the isolator 2 during use is filtered with a HEPA filter 10, thereby removing harmful toxic materials. All air entering the isolator 2 during use may also be filtered with a HEPA filter 10.

As can be seen in FIG. 1, a coupling member 12, disposed around an opening 16, is provided in a side 14 of the isolator 2. As discussed below, the coupling member 12 allows a functional unit 36 to be reversibly connected to the side 14 of the isolator 2.

The coupling member 12 comprises a collar 18 defining an internal surface 20, disposed adjacent to the opening 16, an external surface 22, spaced apart from the opening 16, and an interconnecting edge 24 which extends between the internal and external surfaces. As can be seen in FIG. 5, a vacuum inflatable seal 26 is disposed on the internal surface 20 of the collar 18. Furthermore, an O-ring seal 28 is disposed on the interconnecting edge 24 of the collar 18.

The coupling member 12 further comprises four spaced apart pins 30, each disposed on the interconnecting edge 24 of the collar. The pins 30 are disposed between the O-ring seal 28 and the external surface 22 of the collar 18.

Also disposed on the collar 18 are four spaced apart rotating manual cam locks 32. The rotating manual cam locks 32 are disposed on the interconnecting edge 24, adjacent to external surface 22, of the collar 18.

Finally, the coupling member 12 comprises two pneumatic cylinders 34. Each pneumatic cylinder 34 is disposed adjacent to the external surface 22 of the collar 18, and the two pneumatic cylinders 34 are disposed on opposite sides of the collar 18. Each pneumatic cylinder comprises a bolt 35.

Various functional units 36 are configured to attach to the coupling member 12. For instance, FIGS. 2 to 8 show an embodiment where a blank end cap 36a is fitted to the coupling member 12. Some alternative functional units 36 are shown in FIGS. 9 to 11 and include a rapid transfer port (RTP) 36b, a pass-through hatch end airlock 36c, a centre isolator to isolator pass hatch airlock 36d, an isolator to isolator sleeve 36e, a flexible isolator connector 36f and an isolator to isolator sleeve with a bag out port 36g. It may be appreciated that a user could select a suitable functional unit 36 for a given application.

As shown in FIG. 4, each functional unit 36 comprises a flange 38 configured to fit over at least a portion of the interconnecting edge 24 of the collar 18. Four apertures are provided in the flange 38, and correspond to the locations of the pins 30. Accordingly, when a user is positioning a functional unit 36 on the coupling member 12, either manually or using a cart, the pins 30 and corresponding apertures guide the functional unit 36 into the correct position.

As can be seen in FIG. 5, the functional unit 36 further comprises a collar 40 on a first side of the flange 38. When the functional unit 36 is coupled to the isolator ₂, the collar 40 of the functional unit is configured to fit within and the isolator collar 18. Due to the location of the vacuum inflatable seal 26 between the collars 18, 40 it will create a first seal when it is located. Similarly, when the functional unit 36 is positioned on the isolator 2, the flange 38 is configured to contact the O-ring seal 28, and thereby create a second seal. The second seal is provided in case the first seal should fail. This ensures that, in use, a seal is always provided between the coupling member 12 and functional unit 36.

As can be seen in FIG. 6, four depressions 42 are defined in the flange 38. The depressions 42 are positioned in the flange 38 such that when the functional unit 36 is positioned on the coupling member 12, each depression 42 is located adjacent to a corresponding rotating manual cam lock 32. Accordingly, a user can rotate each of the rotating manual cam locks 32 to manually lock the functional unit 36 into place.

A sensor (not shown) disposed in the isolator 2 senses when the functional unit 36 is coupled to the isolator and manually locked in place. The sensor will send this information to a controller, and the controller will then automatically cause the pneumatic cylinders 34 to move their bolts 35 into corresponding recesses 44 in the flange 38, thereby locking the functional unit 36 to the coupling member 12. The controller will not disengage the pneumatic cylinders 34 until an operator confirms that the isolator 2 has been fully cleaned. This prevents a functional unit 36 from being removed before the isolator 2 has been cleaned.

The controller may then inflate the inflatable seal 26 and initiate an isolator fan (not shown) to cause the pressure within the work area 4 to be reduced. In embodiments where the functional unit 36 is a blank end cap 36a or RTP end cap 36b, the isolator is then ready for use.

Alternatively, in embodiments where the functional unit 36 is a pass-through hatch end airlock 36c, additional sensors within the pass-through hatch end airlock 36c will need to be connected to the controller. Furthermore, the pass-through hatch end airlock 36c will need to be connected to pneumatic air to enable inflatable door seals therein to be inflated. Once these steps have been conducted, in addition to the ones listed above, the isolator will be ready to use.

If the functional unit 36 is am isolator to isolator sleeve 36e then it will need to be connected to another isolator prior to use. This may be achieved using the steps set out above.

Finally, if the functional unit 36 is a centre isolator to isolator pass hatch airlock 36d then the additional steps described for both the pass-through hatch end airlock 36c and the isolator to isolator sleeve 36e will need to conducted prior to use.

After the isolator has been used and fully cleaned, the user must input this information to the controller. The controller will then stop the isolator fan and deflate the vacuum seal 26. A vacuum may be applied to the vacuum seal 26 to ensure it is in the undocked position to prevent any damage. The controller will then disengage the pneumatic cylinders 34. If required, a user will have to disconnect any sensors and pneumatic lines from the functional unit 36 which are connected to the controller. The user can then release the four rotating manual cam locks 32 and remove the functional unit 36 from the coupling member 12.

1. A modular isolator apparatus comprising:
an isolator comprising a housing, having an opening therein, and an isolator coupling member disposed around the opening;
at least one functional unit comprising a functional unit coupling member configured to reversibly couple with the isolator coupling member, thereby reversibly coupling the functional unit and the isolator;
at least one sealing member, configured to create an airtight seal between the isolator and functional unit coupling members when they are coupled; and
at least one locking member, configured to secure the functional unit to the isolator when the isolator and functional unit are coupled;
characterised in that when the functional unit is not secured to the isolator it is removable therefrom.

2. The modular isolator apparatus according to claim 1, wherein the at least one locking member comprises at least one first lock, wherein the or each first lock is configured to be controlled by a user; optionally wherein the at least one locking member comprises a plurality of spaced apart first locks, and the plurality of spaced apart first locks consists of between 2 and 10 first locks.

3. (canceled)

4. The modular isolator apparatus according to claim 2, wherein the or each first lock is a cam lock and is disposed on the isolator.

5. The modular apparatus according to claim 2, wherein the at least one locking member comprises at least one second lock, wherein the apparatus comprises an isolator controller, and the isolator controller is configured to control the or each second lock; optionally wherein the at least one locking member comprises a plurality of spaced apart second locks, and the plurality of spaced apart second locks consists of between 2 and 10 second locks.

6. (canceled)

7. The modular isolator apparatus according to claim 5, wherein the or each second lock is a pneumatic lock and is disposed on the isolator.

8. The modular isolator apparatus according to claim 5, wherein the apparatus comprises at least one sensing units connected to the isolator controller, wherein the at least one sensing units are configured to sense when the functional unit is coupled to the isolator and/or to sense when the or each first lock is locked.

9. The modular isolator apparatus according to claim 8, wherein the isolator controller is configured to engage the at least one second lock when the at least one sensing units sense that the functional unit is coupled to the isolator and/or the or each first lock is locked.

10. The modular isolator apparatus according to 9 claim 5, wherein the isolator controller is configured to not disengage the at least one second lock until a user input confirms that the isolator has been fully cleaned.

11. The modular isolator apparatus according to claim 1, wherein the isolator coupling member comprises an isolator collar, wherein the isolator collar comprises a first side, configured to contact a first side of the functional unit coupling member, a second, internal side, disposed substantially adjacent to the opening; optionally wherein the functional unit coupling member comprises a functional unit collar disposed on the first side of the functional unit coupling member; and/or wherein the functional unit collar is configured to sit within the isolator collar.

12. (canceled)

13. (canceled)

14. The modular isolator apparatus according claim 11, wherein the apparatus comprises a first sealing member, which is disposed on an internal side of the isolator collar.

15. The modular isolator apparatus according to claim 14, wherein the first sealing member is an inflatable seal or vacuum inflatable seal, wherein the apparatus comprises an isolator controller, and the isolator controller is configured to inflate or deform the seal.

16. The modular isolator apparatus according to claim 11, wherein a second sealing member is disposed on the first side of the isolator coupling member, and is preferably an O-ring.

17. The modular isolator apparatus according to claim 1, wherein the apparatus comprises an isolator controller and the isolator controller is configured to power down the apparatus, wherein the isolator controller is configured to not initiate powering down of the apparatus until a user has confirmed that the isolator has been cleaned.

18. The modular isolator apparatus according to claim 1, wherein the apparatus comprises at least one guide member configured to guide the functional unit into a desired position on the coupling member.

19. The modular isolator apparatus according to claim 1, wherein the at least one functional unit comprises a functional unit comprising a panel and the functional unit coupling member is disposed around the edge of the panel.

20. The modular isolator apparatus according to claim 19, wherein the functional unit comprises one or more of a rapid transfer port (RTP), a bag-out port and/or a flexible isolator port disposed in the panel.

21. The modular isolator apparatus according to claim 1, wherein the at least one functional unit comprises a pass-through hatch end airlock functional unit comprising a housing defining a chamber therein, wherein the chamber is configured to allow a user access to the chamber from outside of the isolator.

22. The modular isolator apparatus according to claim 1, wherein the at least one functional unit comprises at least one functional unit configured to reversibly connect the isolator to a second isolator, wherein the at least one functional unit configured to connect the isolator to a second isolator comprises a first functional unit coupling member configured to reversibly couple with a first isolator coupling member on a first isolator, and a second functional unit coupling member configured to reversibly couple with a second isolator coupling member on the second isolator; optionally wherein the at least one functional unit configured to reversibly connect the first isolator to the second isolator comprises a conduit extending between the first and second isolator coupling members; and/or wherein the functional unit comprises a panel extending across the conduit, and the panel comprises one or more of a rapid transfer port, a bag-out port and/or a HEPA filter.

23. (canceled)

24. (canceled)

25. The modular isolator apparatus according to claim 22, wherein the at least one functional unit configured to reversibly connect the isolator to a second isolator comprises a centre isolator to isolator pass hatch airlock functional unit comprising a housing defining a chamber therein, wherein the chamber is configured to reversibly connect the first and second isolators and to allow a user access to the chamber from outside of the isolators.

26. An isolator comprising:
a housing, having an opening therein, and an isolator coupling member disposed around the opening, the isolator coupling member being configured to couple with a functional unit coupling area, and thereby couple the isolator to a functional unit;
at least one sealing member, disposed on the isolator coupling member and being configured to create an airtight seal between the isolator and functional unit coupling members when they are coupled; and
at least one locking member, configured to secure the functional unit to the isolator when the isolator and functional unit are coupled;
characterised in that the isolator may be disconnected from the functional unit.
